(19) United States
(12) Patent Application Publication
(10) Pub. No.: US 2024/0408059 A1
(43) Pub. Date: Dec. 12, 2024

(54) METHOD OF TREATING ADDICTION

(71) Applicant: Anebulo Pharmaceuticals, Inc., Lakeway, TX (US)

(72) Inventors: Daniel Pawel SCHNEEBERGER, Austin, TX (US); Joseph Fenton LAWLER, Lakeway, TX (US)

(21) Appl. No.: 18/700,232

(22) PCT Filed: Oct. 10, 2022
(86) PCT No.: PCT/US2022/046201

§ 371 Date: Apr. 10, 2024

Related U.S. Application Data
(60) Provisional application No. 63/254,473, filed on Oct. 11, 2021.

(51) Int. Cl. A61K 31/397; A61K 31/00; A61K 31/496; A61K 31/55; A61P 25/30

(52) U.S. Cl. CPC A61K 31/397; A61K 31/496; A61K 31/55; A61K 31/658; A61P 25/30

(57) ABSTRACT

Described herein are methods of combination therapy for treating addictive diseases and disorders with CB1 inhibitors. Further described herein are methods of treating addiction with CB1 inhibitors in combination with an antidepressant and/or patient monitoring. Further described herein are methods of treating cannabis use disorder (CUD).

### CROSS-REFERENCE TO RELATED APPLICATIONS

This application claims the benefit of U.S. Provisional Application No. 63/254,473 filed Oct. 11, 2021, which is incorporated herein by reference for all purposes in its entirety.

### BACKGROUND

Addiction represents a serious public health challenge with both physiological and psychological components. Therefore, a medical need exists for new methods to treat addiction.

### SUMMARY OF THE INVENTION

Provided herein are methods of treating addiction with pharmaceutical compositions. Further provided herein are pharmaceutical compositions comprising CB1 inhibitors. Further provided herein are methods of treating addiction with CB1 inhibitors in combination with a second treatment. Further provided are methods wherein the second treatment comprises administering a pharmaceutical or monitoring a patient.

In a first aspect, the present disclosure provides a method of treating an addictive disorder comprising administering to a patient:
(i) a first treatment comprising a pharmaceutical composition, wherein the pharmaceutical composition comprises an effective amount of a CB1 inhibitor; and
(ii) a second treatment.

In some embodiments, the addictive disorder comprises a substance addiction. In some embodiments, the addictive disorder comprises a behavioral addiction. In some embodiments, the addictive disorder comprises addiction to one or more of cannabis, alcohol, nicotine, opioids, amphetamines, cocaine, gambling, or a combination thereof. In some embodiments, the addictive disorder comprises cannabis use disorder (CUD). In some embodiments, the addictive disorder comprises cannabis hyperemesis syndrome (HES). In some embodiments, the second treatment comprises one or more of an antidepressant, CB1 agonist, CB1 modulator, and patient monitoring. In some embodiments, the patient monitoring comprises use of a software application. In some embodiments, the patient monitoring comprises evaluation by a healthcare professional. In some embodiments, the software application is configured for use on mobile phones, tablets, watches or wristbands. In some embodiments, the software application is configured to evaluate the patient's mood or mental state. In some embodiments, the software application is configured to evaluate the patient's vital signs, speech, movement, or sleep patterns. In some embodiments, the software application comprises a patient questionnaire. In some embodiments, the software application comprises monitoring the patient's vital signs. In some embodiments, the CB1 agonist comprises THC or other agonist derived from the cannabis plant. In some embodiments, the CB1 modulator comprises cannabidiol, Org27569 PAM1 (GAT211), ZCZ011, ABD1027, PSNCBAM-1, GAT358, Lipoxin A4, Pregnenolone, or Pepcan 12. In some embodiments, the antidepressant comprises a selective serotonin reuptake inhibitor (SSRI), a serotonin and norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine-dopamine reuptake inhibitor (NDRI), a monoamine oxidase inhibitor (MAOI), a tetracyclic antidepressant (TeCA), an atypical antipsychotic, a tricyclic antidepressant (TCA), an alternative antidepressant, or an over-the-counter antidepressant. In some embodiments, the SSRI comprises citalopram, escitalopram, fluoxetine, fluvoxamine, paroxetine, or sertraline. In some embodiments, the SNRI comprises desvenlafaxine, duloxetine, levomilnacipran, milnacipran, or venlafaxine. In some embodiments, the SMS comprises vilazodone or vortioxetine. In some embodiments, the SARI comprises nefazodone or trazodone. In some embodiments, the NRI comprises reboxetine, teniloxazine, viloxazine, or atomoxetine. In some embodiments, the MAOI comprises isocarboxazid, nialamide, phenelzine, hydracarbazine, tranylcypromirie, bifemelane, moclobemide, pirlindole, toloxatone, rasagiline, selegilin, caroxazone, or safinamide. In some embodiments, the TeCA comprises amoxapine, maprotiline, mianserin, mirtazapine, or setiptiline. In some embodiments, the TCA comprises amitriptyline, amitriptylinoxide, clomipramine, desipramine, dibenzepin, dimetacrine, dosulepin, doxepin, imipramine, lofepramine, melitracen, nitroxazepine, nortriptyline, noxiptiline, opipramol, pipofezine, protriptyline, or trimipramine. In some embodiments, the atypical antipsychotic comprises amisulpride, lurasidone, or quetiapine. In some embodiments, the alternative antidepressant comprises agomelatine, ketamine, tandospirone, tianeptine, and minocycline. In some embodiments, the antidepressant and CB1 inhibitor are metabolized by different enzymes in-vivo. In some embodiments, the CB1 inhibitor is not an inhibitor of CYP3A4. In some embodiments, the CB1 inhibitor is metabolized by one or more of CYP3A4, CYP2C9, CYP2C19, CYP1A2, CYP2E1, CYP2D6, and CYP2A6. In some embodiments, the CB1 inhibitor comprises an IC₅₀ of individual CYP450s of less than 40 micromolar in human liver microsomes. In some embodiments, the antidepressant is metabolized primarily by CYP3A4. In some embodiments, the dose of the antidepressant is between about 0.1 mg and about 1000 mg. In some embodiments, the dose of the antidepressant is between about 15 mg and about 45 mg. In some embodiments, the CB1 inhibitor comprises antagonist, inverse agonist, or reverse agonist. In some embodiments, the CB1 inhibitor has a half-life of at least 2 hours. In some embodiments, the CB1 inhibitor has a half-life of at least 8 hours. In some embodiments, the CB1 inhibitor has a reduced seizure liability. In some embodiments, the CB1 inhibitor comprises rimonabant, taranabant, MK-0364, AM251, AM1387, AM4113, cannabigerol, ibipinabant, otenabant, surinabant, tetrahydrocannabivarin and virodamine, TM-38837, AM6545, or a CB1 targeting-antibody. In some embodiments of the method, the CB1 inhibitor is a compound according to formula (I):

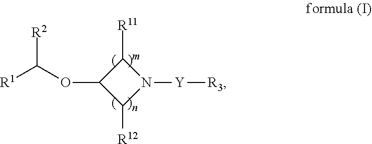

or a pharmaceutically acceptable salt or prodrug thereof,
wherein:
   R¹ is aryl or heteroaryl;
   R² is alkyl, aryl or heteroaryl;
   R³ is alkyl, aryl, heteroaryl, NR⁹R¹⁰, OR¹⁵, or NR¹⁶C(O)R¹⁷;
   Y is C═O, C═S, SO₂, or (CR⁷R⁸)ₚ;
   R⁷ and R⁸ are independently selected from H and lower alkyl;
   R⁹ is selected from H, alkyl, aryl, heteroaryl, and non-aromatic heterocyclic groups, or together with R¹⁰ forms a saturated 4, 5, 6, or 7 membered ring optionally containing an additional heteroatom selected from N and O;
   R¹⁰ is selected from H and lower alkyl, or together with R⁹ forms a saturated 4, 5, 6, or 7 membered ring optionally containing an additional heteroatom selected from N and O;
   R¹¹ and R¹² are independently selected from H and lower alkyl;
   R¹⁵ is selected from alkyl and aryl;
   R¹⁶ is selected from H and lower alkyl;
   R¹⁷ is selected from alkyl, aryl and heteroaryl;
   m is 1 or 2;
   n is 1 or 2; and
   p is 1, 2, 3 or 4. In some embodiments of the method, m is 1 and n is 1. In some embodiments of the method, R¹ and R² are independently aryl. In some embodiments of the method, R¹ and R² has non-hydrogen substituent in the ortho-position(s) thereof relative to the point of attachment to the [—CH—O—] group. In some embodiments of the method, R¹¹ and R¹² are hydrogen. In some embodiments of the method, R³ is NR⁹R¹⁰, and R⁹ and R¹⁰ are independently lower alkyl or hydrogen. In some embodiments of the method, the CB1 inhibitor comprises the structure of formula (Ia):

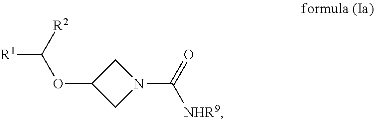

or a pharmaceutically acceptable salt or prodrug thereof. In some embodiments of the method,
R¹ and R² are independently selected from a group of formula (II):

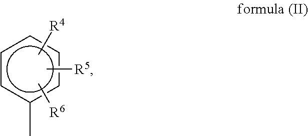

wherein:
   R⁴, R⁵, and R⁶ are independently selected from hydrogen, halo, alkyl (including haloalkyl), thioalkyl, alkoxy (including haloalkoxy), alkylsulfonyl, amino, mono- and di-alkyl amino, mono- and di-aryl amino, alkylarylamino, aminoalkyl, alkylaminoalkyl, dialkylaminoalkyl, NR¹⁴C(O)R¹⁹, NR¹⁴SO₂R²⁰, COOR¹⁹, OC(O)R²⁰, CONR¹³R¹⁴ and SO₂NR¹³R¹⁴,
   R¹³ and R¹⁴ are independently selected from hydrogen and alkyl or form a 5 or 6 membered ring optionally containing 1 or 2 additional heteroatoms selected from N, O and S;
   R¹⁹ is selected from H, alkyl, aryl and heteroaryl, and
   R²⁰ is selected from alkyl, aryl and heteroaryl. In some embodiments of the method, at least one of R⁴, R⁵, and R⁶ are chloro or trifluoromethyl. In some embodiments of the method, the CB1 inhibitor is compound 1, N-(tert-butyl)-3-((4-chlorophenyl)(2-(trifluoromethyl)phenyl)methoxy)azetidine-1-carboxamide, according to the formula:

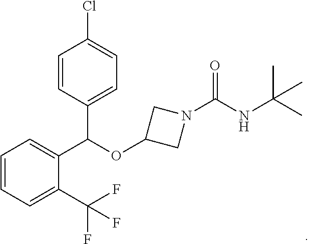 [SMILES: CC(C)(C)NC(=O)N1CC(OC(c2ccc(Cl)cc2)c2ccccc2C(F)(F)F)C1]

In some embodiments of the method, the CB1 inhibitor is compound 2, (R)—N-(tert-butyl)-3-((4-chlorophenyl)(2-(trifluoromethyl)phenyl)methoxy)azetidine-1-carboxamide, according to the formula:

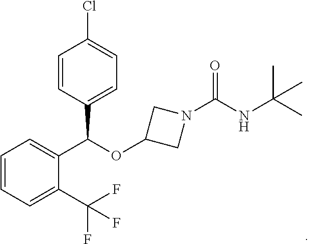 [SMILES: CC(C)(C)NC(=O)N1CC(O[C@H](c2ccc(Cl)cc2)c2ccccc2C(F)(F)F)C1]

In some embodiments of the method, the CB1 inhibitor is compound 3, (S)—N-(tert-butyl)-3-((4-chlorophenyl)(2-(trifluoromethyl)phenyl)methoxy)azetidine-1-carboxamide, according to the formula:

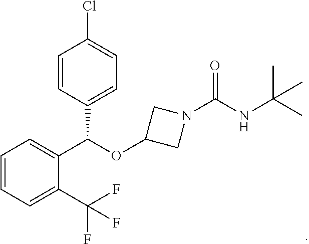 [SMILES: CC(C)(C)NC(=O)N1CC(O[C@@H](c2ccc(Cl)cc2)c2ccccc2C(F)(F)F)C1]

In some embodiments of the method, the dose of the CB1 inhibitor is between about 1 mg and about 200 mg. In some embodiments of the method, the dose of the CB1 inhibitor is between about 1 mg and about 50 mg. In some embodiments of the method, the dose of the CB1 inhibitor is between about 20 mg and about 30 mg. In some embodiments, the dose of the CB1 inhibitor is between about 5 mg and about 100 mg. In some embodiments, the dose of the CB1 inhibitor is between about 10 mg and about 90 mg. In some embodiments, the dose of the CB1 inhibitor is between about 15 mg and about 80 mg. In some embodiments, the dose of the CB1 inhibitor is between about 20 mg and about 70 mg. In some embodiments, the dose of the CB1 inhibitor is between about 30 mg and about 60 mg. In some embodiments, the dose of the CB1 inhibitor is between about 40 mg and about 50 mg. In some embodiments of the method, the CB1 inhibitor is formulated for oral, parenteral, intravenous (IV), intramuscular (IM), subcutaneous (SC), endotracheal, sublingual, buccal, intralingual, submental, transdermal, suppository, or intranasal administration. In some embodiments, the method further comprising administering a pharmaceutically acceptable alkaline agent. In some embodiments of the method, the pharmaceutical composition is formulated to deliver an effective dose of the CB1 inhibitor in no more than 10 minutes. In some embodiments, the CB1 inhibitor is dosed daily. In some embodiments, the antidepressant is dosed daily. In a second aspect, the present disclosure provides a pharmaceutical composition comprising the CB1 inhibitor as described herein and an antidepressant as described herein. In some embodiments, the pharmaceutical composition is formulated as a capsule or a tablet.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a non-limiting example of a web/mobile application provision system for patient monitoring; in this case, a system providing browser-based and/or native mobile user interfaces. The system in some instances allows a patient being treated for addiction using the methods described herein to share health data with a healthcare provider.

### DETAILED DESCRIPTION OF THE INVENTION

Described herein are compositions, formulations, methods, and devices for treating addiction. Treatments in some instances comprise administering a CB1 inhibitor (e.g., compound) and providing a second treatment such as administering a second pharmaceutical or monitoring the patient. Further provided are methods wherein the second treatment comprises administering an antidepressant. Other aspects of the compositions and methods described herein are described below or throughout this specification.

### Methods of Treatment

Provided herein are methods of treating a patient with a pharmaceutical composition comprising a CB1 inhibitor and a second treatment. In some instances, the patient has an addictive disorder, such as a substance abuse addiction or a behavioral addiction. The second treatment in some instances comprises patient monitoring and/or treatment with antidepressants. In some embodiments, the second treatment comprises one or more of an antidepressant, CB1 agonist, CB1 modulator, and patient monitoring. Accordingly, in another aspect, the present disclosure provides a method of treating an addictive disorder comprising administering to a patient: (i) a first treatment comprising a pharmaceutical composition, wherein the pharmaceutical composition comprises an effective amount of a CB1 inhibitor; and (ii) a second treatment.

An addictive behavior treated herein may comprise a substance abuse addiction. In some embodiments, the addictive disorder comprises addiction to one or more of cannabis, alcohol, nicotine, opioids, amphetamines, cocaine, or a combination thereof.

A treatment described herein may be applied to a patient in need thereof to treat a cannabis-related addiction or disorder. In some embodiments, the addictive disorder comprises cannabis use disorder (CUD). In some embodiments, the addictive disorder comprises cannabis hyperemesis syndrome (HES). Cannabis Use Disorder (CUD). There are between about 50 to 60 million cannabis users in the United States and approximately 10% develop Cannabis Use Disorder (CUD). CUD can have severe consequences with regards to educational and career achievements, as in some studies only 50% of patients with CUD are employed or in school. In some instances, a method describes diagnosing a patient with CUD. In some instances, diagnosis is made if a patient comprises one, two, three, four, five or more of the symptoms described in the DSM-5. According to the Diagnostic and Statistical Manual of Mental Disorders Fifth Edition (DSM-5), the criteria for CUD include, but is not limited to, the following:
Use of cannabis for at least a one-year period, with the presence of at least two of the symptoms described herein, accompanied by significant impairment of functioning and distress;
Difficulty containing use of cannabis—the drug is used in larger amounts and over a longer period than intended;
Repeated failed efforts to discontinue or reduce the amount of cannabis that is used
An inordinate amount of time is occupied acquiring, using, or recovering from the effects of cannabis;
Cravings or desires to use cannabis. This can include intrusive thoughts and images, and dreams about cannabis, or olfactory perceptions of the smell of cannabis, due to preoccupation with cannabis;
Continued use of cannabis despite adverse consequences from its use, such as criminal charges, ultimatums of abandonment from spouse/partner/friends, and poor productivity;
Other important activities in life, such as work, school, hygiene, and responsibility to family and friends are superseded by the desire to use cannabis;
Cannabis is used in contexts that are potentially dangerous, such as operating a motor vehicle;
Use of cannabis continues despite awareness of physical or psychological problems attributed to use—e.g., anergia, amotivation, chronic cough;
Tolerance to Cannabis, as defined by progressively larger amounts of cannabis are needed to obtain the psychoactive effect experienced when use first commenced, or, noticeably reduced effect of use of the same amount of cannabis; and
Withdrawal, defined as the typical withdrawal syndrome associate with cannabis, or cannabis or a similar substance is used to prevent withdrawal symptoms.

The severity of the disorder is also noted, depending on the number of symptoms noted as:
Mild—two or three symptoms
Moderate—four or five symptoms
Severe—six or more symptoms, according to the American Psychiatric Association, 2013.

Drugs that block a receptor or prevent its full activation in some instances are useful treatment adjuncts because the patient is unable to get high or experience euphoria. Examples include, but are not limited to, narcan and methadone for narcotic addiction. Antidipsotropic drugs are useful in the treatment of alcoholism because they precipitate illness in the individual if they consume alcohol while on the antidipostropic drug. In some instances, CB1 inhibitors described herein are useful for blocking CB1 receptors associated with addiction. In some instances, CB1 inhibitors described herein are useful for blocking CB1 receptors associated with CUD.

Methods described herein may be used to treat behavior disorders. Behavioral disorders include but are not limited to gambling, overeating, use of electronic devices, use of electronic video games, use of electronic communication devices, pathological use of cellular telephones, addiction to pornography, sex addiction, obsessive compulsive disorder, compulsive spending, anorexia, bulimia, intermittent explosive disorder, kleptomania, pyromania, trichotillomania, compulsive exercising, and compulsive working. In various embodiments of the present disclosure, the subject is suffering from a non-substance addiction. In some embodiments, the non-substance addition is a behavioral addiction. In some embodiments, the non-substance addiction comprises gambling, risky sex, good, the internet, mobile devices, and shopping. In some embodiments, the symptoms include, but are not limited to, compulsive, excessive, and repetitive engagement in a risky behavior or activity, inability to stop or control said risky activity or behavior, impaired judgment, craving said risky behavior or activity, inability to recognize the consequences of the behavior, strained relationships, and inappropriate emotional response.

### Patient Monitoring

Provided herein are systems and methods comprising patient monitoring. In some instances, a patient suffering addiction is treated with a CB1 inhibitor described herein and monitored. In some instances, a patient is also treated with a CB1 agonist, CB1 modulator, or antidepressant concurrently. In some instances patient monitoring is useful for detecting changes in mood which could lead to depression or suicidal ideation. In some embodiments, the patient monitoring comprises use of a software application. In some embodiments, the patient monitoring comprises evaluation by a healthcare professional. In some embodiments, the software application is configured for use on mobile phones, tablets, watches or wristbands (producing devices). In some embodiments, producing devices obtain sensor data. In some embodiments, the software application is configured to evaluate the patient's mood or mental state. In some embodiments, the software application is configured to evaluate the patient's vital signs, speech, movement, or sleep patterns. In some embodiments, the software application comprises a patient questionnaire. In some embodiments, the software application comprises monitoring the patient's vital signs. In some instances, healthcare providers receive alerts or are able to review patient data using the methods and systems described herein. In some instances, a software application comprises a user interface for emergency contact (911 service or on-call healthcare provider).

A user (e.g., patient or healthcare provider) may provide credentials to access a monitoring system such as biometric or password data. In some instances, credentials are linked to one or more device identifiers to provide enhanced security and privacy to the user. In some embodiments, a patient registers or confirms their pre-loaded patient information, such as name, age, mobile phone number, address, physician Information, electronic medical record, or other personal information via the software application. In some embodiments, the registration feature is provided in a software module of the application. In a cloud-based system, the registration module may be provided via a web based application. The patient information may be confirmed or registered with an electronic medical record. In some embodiments, a patient registers or confirms their pre-loaded patient information via a phone call. The phone call may be a 3-way phone call. The 3-way phone call may include the patient, a medical technician, and a physician, licensed, professional, telemedical healthcare provider, or other professional. In some embodiments, a patient registers or confirms their pre-loaded patient information via a text message.

Users may input data into a monitoring system provided herein. In some instances, users manually enter data through a device. In some instances, data is inputted automatically from forms or electronic medical records. In some instances, data is inputted by a patient. In some instances, data comprises health data. In some instances, health data comprises information relating to the patient's mood. In some instances, health data comprises answers to one or more questionnaires. In some instances, the questionnaire measures the patients mood or tendency for depression or suicidal ideation. In some instances, the questionnaire comprises form PHQ-9 (Pfizer) or similar form.

Monitoring systems may comprise a user interface. In some instances, the user interface comprises one or more health reports, report sharing status, reminders, or other interactive interface. In some instances, health reports comprise health data. In some instances, health reports comprise answers to questionnaires and vital/health data. In some instances, reminders comprise instruction to patients to take medication, submit health reports, or requests from healthcare providers to schedule an appointment. In some instances reminders comprise instructions to healthcare professionals to review patient-submitted health reports. In some instances, report sharing status indicates if a report has been completed by a patient, submitted to a healthcare professional, and/or reviewed by the healthcare professional.

In some embodiments, a producing device is a wearable device (e.g., a communication device worn by a user, such as an Apple Watch). In some embodiments, a producing device (e.g., a wearable device) comprises one or more sensors. The one or more sensors optionally include, but are not limited to: a gyroscope, an accelerometer, a thermometer, a heart rate sensor, a barometer, or a hematology analyzer. As used herein, a “mobile wireless device” refers to a device that is portable and communicates wirelessly. In some embodiments, a user wears or carries the mobile wireless device on the user's person or in the user's vehicle. Non-limiting examples of mobile wireless devices include mobile or cellular phones, wearable devices (e.g., smart watch, fitness tracker, wearable sensor, smart glasses, etc.).

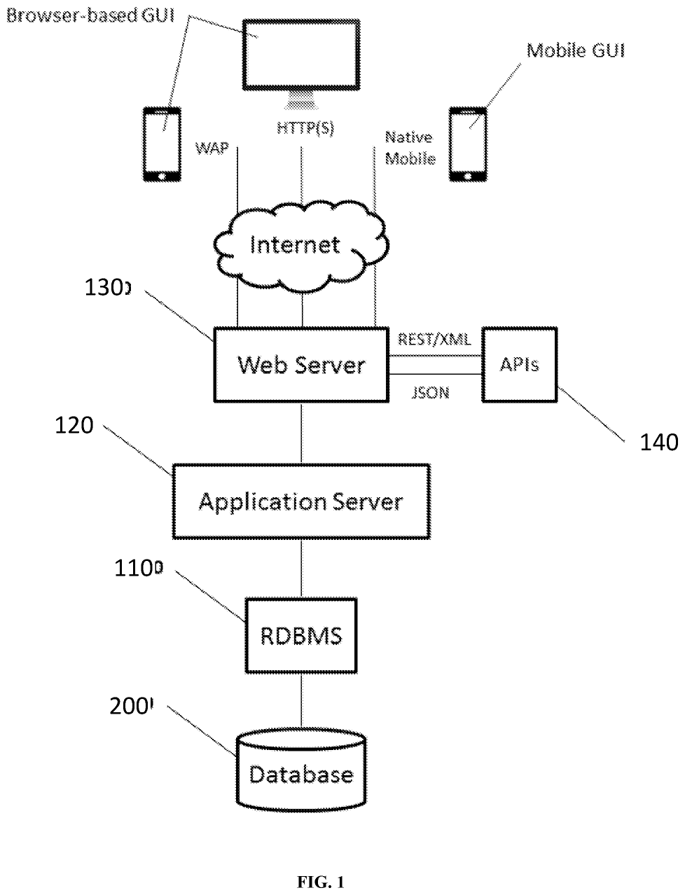

Referring to FIG. 1, in a particular embodiment, a monitoring system comprises one or more databases 100 accessed by a relational database management system (RDBMS) 110. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 120 (such as Java servers, NET servers, PUP servers, and the like) and one or more web servers 130 (such as Apache, IIS, GWS and the like). The web server(s) optionally expose one or more web services via app application programming interfaces (APIs) 140. Via a network, such as the Internet, the system provides browser-based and/or mobile native user interfaces.

### Combination Treatment

Provided herein are methods of treating addiction comprising administration of a pharmaceutical composition comprising a CB1 inhibitor and a second treatment.

While many drugs have been tried for treatment for CUD, including Mirtazapine monotherapy, but to date no successful treatment has been developed. CUD in some instances is treated both acutely and in the long term by antagonizing the same receptor. Some previous studies with CB1 antagonists have shown there is a small but detectable rise in the incidence of depression amongst patients taking the drug long term (9% vs 5% with Rimonabant). There has also been a potential increase in suicide and suicide ideation, with hazard ratios of 1.3 (according to Sanofi) to 1.9 (according to the FDA).

A second treatment may comprise administering a pharmaceutical. In some instances, the pharmaceutical comprises a CB1 agonist, CB1 modulator, an antidepressant, or a combination thereof. In some embodiments, the CB1 agonist comprises THC or other agonist derived from the cannabis plant. In some embodiments, the CB1 modulator comprises cannabidiol, Org27569 PAM1 (GAT211), ZCZ011, ABD1027, PSNCBAM-1, GAT358, Lipoxin A4, Pregnenolone, or Pepcan 12.

A second treatment may comprise administering an antidepressant. In some embodiments, the antidepressant comprises a selective serotonin reuptake inhibitor (SSRI), a serotonin and norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine-dopamine reuptake inhibitor (NDRI), a monoamine oxidase inhibitor (MAOI), a tetracyclic antidepressant (TeCA), an atypical antipsychotic, a tricyclic antidepressant (TCA), an alternative antidepressant, or an over-the-counter antidepressant. In some embodiments, the SSRI comprises citalopram, escitalopram, fluoxetine, fluvoxamine, paroxetine, or sertraline. In some embodiments, the SNRI comprises desvenlafaxine, duloxetine, levomilnacipran, milnacipran, or venlafaxine. In some embodiments, the SMS comprises vilazodone or vortioxetine. In some embodiments, the SARI comprises nefazodone or trazodone. In some embodiments, the NRI comprises reboxetine, teniloxazine, viloxazine, or atomoxetine. In some embodiments, the MAOI comprises isocarboxazid, nialamide, phenelzine, hydracarbazine, tranylcypromirie, bifemelane, moclobemide, pirlindole, toloxatone, rasagiline, selegilin, caroxazone, or safinamide. In some embodiments, the TeCA comprises amoxapine, maprotiline, mianserin, mirtazapine, or setiptiline. In some embodiments, the TCA comprises amitriptyline, amitriptylinoxide, clomipramine, desipramine, dibenzepin, dimetacrine, dosulepin, doxepin, imipramine, lofepramine, melitracen, nitroxazepine, nortriptyline, noxiptiline, opipramol, pipofezine, protriptyline, or trimipramine. In some embodiments, the atypical antipsychotic comprises amisulpride, lurasidone, or quetiapine. In some embodiments, the alternative antidepressant comprises agomelatine, ketamine, tandospirone, tianeptine, and minocycline.

Combinations of CB1 inhibitors and antidepressants may be selected to minimize drug-drug interactions. In some embodiments, the antidepressant and CB1 inhibitor are metabolized by different enzymes in-vivo. In some embodiments, the CB1 inhibitor is not an inhibitor of CYP3A4. In some embodiments, the CB1 inhibitor is metabolized by one or more of CYP3A4, CYP2C9, CYP2C19, CYP1A2, CYP2E1, CYP2D6, and CYP2A6. In some embodiments, the CB1 inhibitor comprises an IC₅₀ of individual CYP450s of less than 100, 80, 70, 60, 50, 40, 30, 20, or 10 micromolar in human liver microsomes. In some embodiments, the antidepressant is metabolized primarily by CYP3A4.

Dosages of pharmaceuticals may be adjusted as needed for treatments described herein. In some instances, the pharmaceutical comprises an antidepressant, CB1 agonist, CB1 modulator, or combination thereof. In some embodiments, the dose of the antidepressant is 0.1-1000 mg, 0.1-500 mg, 0.1-250 mg, 0.1-100 mg, 0.1-50 mg, 0.1-25 mg, 0.1-10 mg, 0.1-5 mg, 0.1-1 mg, 0.5-10 mg, 1-50 mg, 1-100 mg, 1-1000 mg, 10-1000 mg, 15-1000, 15-45 mg, 25-50 mg, 25-100 mg, 50-1000 mg, 100-1000 mg, or 500-1000 mg. In some embodiments, the dose of the CB1 agonist is 0.1-1000 mg, 0.1-500 mg, 0.1-250 mg, 0.1-100 mg, 0.1-50 mg, 0.1-25 mg, 0.1-10 mg, 0.1-5 mg, 0.1-1 mg, 0.5-10 mg, 1-50 mg, 1-100 mg, 1-1000 mg, 10-1000 mg, 15-1000, 15-45 mg, 25-50 mg, 25-100 mg, 50-1000 mg, 100-1000 mg, or 500-1000 mg. In some embodiments, the dose of the CB1 modulator is 0.1-1000 mg, 0.1-500 mg, 0.1-250 mg, 0.1-100 mg, 0.1-50 mg, 0.1-25 mg, 0.1-10 mg, 0.1-5 mg, 0.1-1 mg, 0.5-10 mg, 1-50 mg, 1-100 mg, 1-1000 mg, 10-1000 mg, 15-1000, 15-45 mg, 25-50 mg, 25-100 mg, 50-1000 mg, 100-1000 mg, or 500-1000 mg.

### Pharmaceutical Compositions

In another aspect, the present disclosure provides compositions, including pharmaceutical composition, comprising a CB1 inhibitor, and at least one pharmaceutically acceptable excipient. In some embodiments, the pharmaceutical composition further comprises an antidepressant.

In some embodiments, the pharmaceutical composition is prepared as an oral, a sublingual, a buccal, a rectal, a nasal, or a parenteral dose. In some embodiments, the dose of the CB1 inhibitor is between about 1 mg and about 200 mg. In some embodiments, the dose of the CB1 inhibitor is between about 5 mg and about 100 mg. In some embodiments, the dose of the CB1 inhibitor is 0.1-1000 mg, 0.1-500 mg, 0.1-250 mg, 0.1-100 mg, 0.1-50 mg, 0.1-25 mg, 0.1-10 mg, 0.1-5 mg, 0.1-1 mg, 0.5-10 mg, 1-50 mg, 1-100 mg, 1-1000 mg, 10-1000 mg, 15-1000, 15-45 mg, 20-30 mg, 25-50 mg, 25-100 mg, 50-1000 mg, 100-1000 mg, or 500-1000 mg.

In some embodiments, the pharmaceutical composition is formulated for oral, parenteral, intravenous (IV), intramuscular (IM), subcutaneous (SC), endotracheal, sublingual, buccal, intralingual, submental, transdermal, suppository, or intranasal administration.

In some embodiments, the pharmaceutical composition is formulated to deliver a therapeutically effective dose of a CB1 inhibitor in no more than 8 hours, 6 hours, 4 hours, 2 hours, 1 hour, 45 minutes, 30 minutes, 20 minutes or no more than 10 minutes.

In some embodiments, the pharmaceutical composition comprising a CB1 inhibitor is formulated as a capsule or table.

In another aspect, the present disclosure provides a pharmaceutical composition comprising a CB1 inhibitor (e.g., compound 2) as described herein and an antidepressant. In some embodiments, the antidepressant comprises the antidepressant comprises a selective serotonin reuptake inhibitor (SSRI), a serotonin and norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine-dopamine reuptake inhibitor (NDRI), a monoamine oxidase inhibitor (MAOI), a tetracyclic antidepressant (TeCA), an atypical antipsychotic, a tricyclic antidepressant (TCA), an alternative antidepressant, or an over-the-counter antidepressant. In some embodiments, the SSRI comprises citalopram, escitalopram, fluoxetine, fluvoxamine, paroxetine, or sertraline. In some embodiments, the SNRI comprises desvenlafaxine, duloxetine, levomilnacipran, milnacipran, or venlafaxine. In some embodiments, the SMS comprises vilazodone or vortioxetine. In some embodiments, the SARI comprises nefazodone or trazodone. In some embodiments, the NRI comprises reboxetine, teniloxazine, viloxazine, or atomoxetine. In some embodiments, the MAOI comprises isocarboxazid, nialamide, phenelzine, hydracarbazine, tranylcypromirie, bifemelane, moclobemide, pirlindole, toloxatone, rasagiline, selegilin, caroxazone, or safinamide. In some embodiments, the TeCA comprises amoxapine, maprotiline, mianserin, mirtazapine, or setiptiline. In some embodiments, the TCA comprises amitriptyline, amitriptylinoxide, clomipramine, desipramine, dibenzepin, dimetacrine, dosulepin, doxepin, imipramine, lofepramine, melitracen, nitroxazepine, nortriptyline, noxiptiline, opipramol, pipofezine, protriptyline, or trimipramine. In some embodiments, the atypical antipsychotic comprises amisulpride, lurasidone, or quetiapine. In some embodiments, the alternative antidepressant comprises agomelatine, ketamine, tandospirone, tianeptine, and minocycline. In some embodiments, the over-the-counter antidepressant comprises ademetionine, Hypericum perforatum, oxitriptan, rubidium chloride, or tryptophan.

In certain embodiments, pharmaceutical compositions are formulated in any conventional manner using one or more physiologically acceptable carriers comprising excipients and auxiliaries which facilitate processing of the active polymorphs into preparations which can be used pharmaceutically. Proper formulation is dependent upon the route of administration chosen. Any pharmaceutically acceptable techniques, carriers, and excipients are optionally used as suitable. Pharmaceutical compositions comprising a CB1 inhibitor as described herein are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one CB1 inhibitor as described herein as an active ingredient. The active ingredient is in acid-free or base-free form, or in a pharmaceutically acceptable salt form. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions, comprising a CB1 inhibitor as described herein include formulating the compound with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutically acceptable carriers or excipients for formulations described herein may comprise one or more CB1 inhibitor. In some embodiments, the pharmaceutically acceptable carrier is a polymer. Examples of polymers suitable for oral, buccal, intranasal, transdermal, thin-film, suppository or other administration include biocompatible and biodegradable polymers. Further examples of biocompatible polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like. In some instances, the carrier is Labrasol. In some instances, the carrier is methyl cellulose. In further embodiments, the pharmaceutically acceptable carrier comprises one or more biodegradable polymers. Use of biodegradable polymers provides the advantages of using a formulation that will eventually disintegrate, which facilitates release of the benzofuran compound and elimination of the carrier in vivo. However, benzofuran compounds can also be released from the matrix of non-biodegradable polymers as a result of gradual efflux from channels within the polymer matrix, including those formed by soluble materials included in the polymer matrix.

Examples of biodegradable polymers include polylactide polymers include poly(D,L-lactide)s; poly(lactide-co-glycolide) (PLGA) copolymers; polyglycolide (PGA) and polydioxanone; caprolactone polymers; chitosan; hydroxybutyric acids; polyanhydrides and polyesters; polyphosphazenes; and polyphosphoesters. In some instances, the biodegradable polymer for use in the nanoparticles is poly-(D,L-lactide-co-glycolide).

Functionalized poly (D,L-lactide)s can also be used as biodegradable polymers in the nanoparticles described herein. Examples of functionalized poly(D,L-lactide)s include poly(L-lactide), acrylate terminated; poly(L-lactide), amine terminated; poly(L-lactide), azide terminated; poly(L-lactide), 2-bromoisobutyryl terminated; poly(L-lactide), 2-bromoisobutyryl terminated; poly(L-lactide) 4-cyano-4-[(dodecylsulfanylthiocarbonyl)sulfanyl]pentonate; poly(L-lactide) N-2-hydroxyethylmaleimide terminated; poly(L-lactide) 2-hydroxyethyl, methacrylate terminated; poly(L-lactide), propargyl terminated; or poly(L-lactide), thiol terminated.

Other biodegradable polymers that can be used in the nanoparticles include AB-15-eblock copolymers such as poly(ethylene glycol) methyl ether-block-poly(D,L-lactide); poly(ethylene glycol) methyl ether-block-poly(lactide-co-glycolide) PEG; poly(ethylene glycol)-block-poly(.epsilon.-caprolactone) methyl ether PEG; and polypyrrole-block-poly(caprolactone). Further biodegradable polymers include ABA triblock copolymers such as polylactide-block-poly(ethylene glycol)-block-polylactide PLA; poly(lactide-co-glycolide)-block-poly(ethylene glycol)-block-poly(lactide-co-glycolide); poly(lactide-co-caprolactone)-block-poly(ethylene glycol)-block-poly(lactide-co-caprolactone); polycaprolactone-block-polytetrahydrofuran-block-polycaprolactone; and polyglycolide-block-poly(ethylene glycol)-block-polyglycolide PEG.

Biodegradable polymers also include various natural polymers. Examples of natural polymers include polypeptides including those modified non-peptide components, such as saccharide chains and lipids; nucleotides; sugar-based biopolymers such as polysaccharides; cellulose; carbohydrates and starches; dextrans; lignins; polyamino acids; adhesion proteins; lipids and phospholipids (e.g., phosphorylcholine). In some embodiments, the polymer is a cellulose derivative such as hydroxypropyl methylcellulose polymers. Hydroxypropyl methyl cellulose (HPMC) is a non-ionic cellulose ether made through a series of chemical processes, with the natural polymer cellulose as the raw material. The product is a non-ionic cellulose ether in the shape of white powder, odorless and tasteless. HPMC is also known as hypromellose, is a methylcellulose modified with a small amount of propylene glycol ether groups attached to the anhydroglucose of the cellulose.

Useful pharmaceutical compositions also, optionally, include solubilizing agents to aid in the solubility a CB1 inhibitor. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Pharmaceutical compositions described herein may comprise CB1 inhibitors. In some embodiments, the CB1 inhibitor comprises antagonist, inverse agonist, or reverse agonist.

In some embodiments, the CB1 inhibitor comprises rimonabant, taranabant, MK-0364, AM251, AM1387, AM4113, cannabigerol, ibipinabant, otenabant, surinabant, tetrahydrocannabivarin and virodamine, TM-38837, AM6545, or a CB1 targeting-antibody.

In some embodiments of the method, the CB1 inhibitor is a compound according to formula (I):

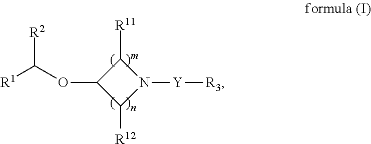

or a pharmaceutically acceptable salt or prodrug thereof,
wherein:
   R¹ is aryl or heteroaryl;
   R² is alkyl, aryl or heteroaryl;
   R³ is alkyl, aryl, heteroaryl, NR⁹R¹⁰, OR¹⁵, or NR¹⁶C(O)R¹⁷;
   Y is C═O, C═S, SO₂, or (CR⁷R⁸)ₚ;
   R⁷ and R⁸ are independently selected from H and lower alkyl;
   R⁹ is selected from H, alkyl, aryl, heteroaryl, and non-aromatic heterocyclic groups, or together with R¹⁰ forms a saturated 4, 5, 6, or 7 membered ring optionally containing an additional heteroatom selected from N and O;
   R¹⁰ is selected from H and lower alkyl, or together with R⁹ forms a saturated 4, 5, 6, or 7 membered ring optionally containing an additional heteroatom selected from N and O;
   R¹¹ and R¹² are independently selected from H and lower alkyl;
   R¹⁵ is selected from alkyl and aryl;
   R¹⁶ is selected from H and lower alkyl;
   R¹⁷ is selected from alkyl, aryl and heteroaryl;
   m is 1 or 2;
   n is 1 or 2; and
   p is 1, 2, 3 or 4.

In some embodiments of the method, m is 1 and n is 1. In some embodiments of the method, R¹ and R² are independently aryl. In some embodiments of the method, R¹ and R² has non-hydrogen substituent in the ortho-position(s) thereof relative to the point of attachment to the [—CH—O—] group. In some embodiments of the method, R¹¹ and R¹² are hydrogen. In some embodiments of the method, R³ is NR⁹R¹⁰, and R⁹ and R¹⁰ are independently lower alkyl or hydrogen. In some embodiments of the method, the CB1 inhibitor comprises the structure of formula (Ia):

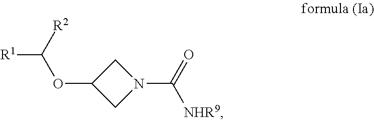

or a pharmaceutically acceptable salt or prodrug thereof. In some embodiments of the method,
R¹ and R² are independently selected from a group of formula (II):

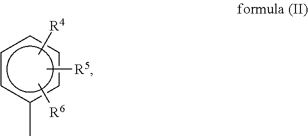

wherein:
   R⁴, R⁵, and R⁶ are independently selected from hydrogen, halo, alkyl (including haloalkyl), thioalkyl, alkoxy (including haloalkoxy), alkylsulfonyl, amino, mono- and di-alkyl amino, mono- and di-aryl amino, alkylarylamino, aminoalkyl, alkylaminoalkyl, dialkylaminoalkyl, NR¹⁴C(O)R¹⁹, NR¹⁴SO₂R²⁰, COOR¹⁹, OC(O)R²⁰, CONR¹³R¹⁴ and SO₂NR¹³R¹⁴,
   R¹³ and R¹⁴ are independently selected from hydrogen and alkyl or form a 5 or 6 membered ring optionally containing 1 or 2 additional heteroatoms selected from N, O and S;
   R¹⁹ is selected from H, alkyl, aryl and heteroaryl, and
   R²⁰ is selected from alkyl, aryl and heteroaryl.

In some embodiments of the method, at least one of R⁴, R⁵, and R⁶ are chloro or trifluoromethyl. In some embodiments of the method, the CB1 inhibitor is compound 1, N-(tert-butyl)-3-((4-chlorophenyl)(2-(trifluoromethyl)phenyl)methoxy)azetidine-1-carboxamide, according to the formula:

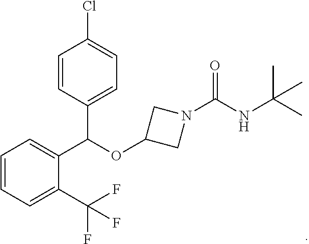 [SMILES: CC(C)(C)NC(=O)N1CC(OC(c2ccc(Cl)cc2)c2ccccc2C(F)(F)F)C1]

In some embodiments of the method, the CB1 inhibitor is compound 2, (R)—N-(tert-butyl)-3-((4-chlorophenyl)(2-(trifluoromethyl)phenyl)methoxy)azetidine-1-carboxamide, according to the formula:

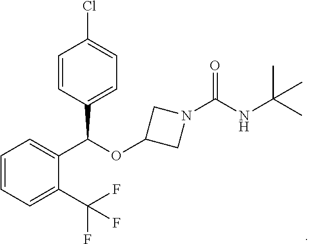 [SMILES: CC(C)(C)NC(=O)N1CC(O[C@H](c2ccc(Cl)cc2)c2ccccc2C(F)(F)F)C1]

In some embodiments of the method, the CB1 inhibitor is compound 3, (S)—N-(tert-butyl)-3-((4-chlorophenyl)(2-(trifluoromethyl)phenyl)methoxy)azetidine-1-carboxamide, according to the formula:

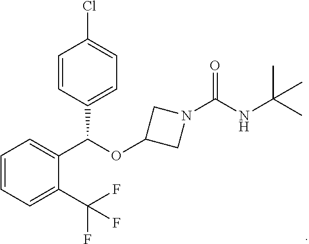 [SMILES: CC(C)(C)NC(=O)N1CC(O[C@@H](c2ccc(Cl)cc2)c2ccccc2C(F)(F)F)C1]

Any dosage may be used for CB1 inhibitors described herein. In some embodiments of the method, the dose of the CB1 inhibitor is between about 1 mg and about 200 mg. In some embodiments, the dose of the CB1 inhibitor is between about 1 mg and about 50 mg. In some embodiments, the dose of the CB1 inhibitor is between about 5 mg and about 100 mg. In some embodiments, the dose of the CB1 inhibitor is between about 10 mg and about 90 mg. In some embodiments, the dose of the CB1 inhibitor is between about 15 mg and about 80 mg. In some embodiments, the dose of the CB1 inhibitor is between about 20 mg and about 70 mg. In some embodiments, the dose of the CB1 inhibitor is between about 30 mg and about 60 mg. In some embodiments, the dose of the CB1 inhibitor is between about 40 mg and about 50 mg. In some embodiments, the dose of the CB1 inhibitor is between about 20 mg and about 30 mg. In some instances, the CB1 inhibitor is compound 2.

Any dosage may be used for pharmaceutical compositions comprising CB1 inhibitors described herein. In some embodiments of the method, the dose of the CB1 inhibitor is between about 1 mg and about 200 mg. In some embodiments, the dose of the CB1 inhibitor is between about 1 mg and about 50 mg. In some embodiments, the dose of the CB1 inhibitor is between about 5 mg and about 100 mg. In some embodiments, the dose of the CB1 inhibitor is between about 10 mg and about 90 mg. In some embodiments, the dose of the CB1 inhibitor is between about 15 mg and about 80 mg. In some embodiments, the dose of the CB1 inhibitor is between about 20 mg and about 70 mg. In some embodiments, the dose of the CB1 inhibitor is between about 30 mg and about 60 mg. In some embodiments, the dose of the CB1 inhibitor is between about 40 mg and about 50 mg. In some embodiments, the dose of the CB1 inhibitor is between about 20 mg and about 30 mg. In some instances, the pharmaceutical composition comprises compound 2.

In some embodiments of the method, the CB1 inhibitor is formulated for oral, parenteral, intravenous (IV), intramuscular (IM), subcutaneous (SC), endotracheal, sublingual, buccal, intralingual, submental, transdermal, suppository, or intranasal administration.

In some embodiments, the method further comprising administering a pharmaceutically acceptable alkaline agent.

In some embodiments of the method, the pharmaceutical composition is formulated to deliver an effective dose of the CB1 inhibitor (e.g., compound 2) in no more than 10 minutes. In some embodiments, the CB1 inhibitor is dosed monthly, weekly, or daily. In some embodiments, the CB1 inhibitor is dosed in combination with another pharmaceutical (e.g., antidepressant, CB1 agonist, or CB1 modulator. In some embodiments, the CB1 inhibitor is dosed in combination with an antidepressant. In some embodiments, the antidepressant is dosed monthly, weekly, or daily.

In various embodiments of the methods described herein, the amount of the CB1 inhibitor (e.g., compound 2) in the bloodstream of the subject reaches at least 200 ng/mL within one hour after oral administration. In another embodiment, the amount of the CB1 inhibitor in the bloodstream of the subject reaches at least 200 ng/mL within 50 minutes after oral administration. In another embodiment, the amount of the CB1 inhibitor in the bloodstream of the subject reaches at least 200 ng/mL within 40 minutes after oral administration. In another embodiment, the amount of the CB1 inhibitor in the bloodstream of the subject reaches at least 200 ng/mL within 30 minutes after oral administration. In another embodiment, the amount of the CB1 inhibitor in the bloodstream of the subject reaches at least 200 ng/mL within 20 minutes after oral administration. In another embodiment, the amount of the CB1 inhibitor in the bloodstream of the subject reaches at least 200 ng/mL within 10 minutes after oral administration. In another embodiment, the amount of the CB1 inhibitor in the bloodstream of the subject reaches at least 200 ng/mL within 5 minutes after oral administration. In some embodiments, the CB1 inhibitor is compound 2.

In some instances, the CB1 inhibitor comprises (R)—N-(tert-butyl)-3-((4-chlorophenyl)(2-(trifluoromethyl)phenyl)methoxy)azetidine-1-carboxamide is between about 0.1 mg to about 200 mg. In another embodiment, the amount of (R)—N-(tert-butyl)-3-((4-chlorophenyl)(2-(trifluoromethyl)phenyl)methoxy)azetidine-1-carboxamide is between about 1 mg to about 200 mg. In another embodiment, the amount of (R)—N-(tert-butyl)-3-((4-chlorophenyl)(2-(trifluoromethyl)phenyl)methoxy)azetidine-1-carboxamide is between about 10 mg to about 50 mg. In another embodiment, the amount of (R)—N-(tert-butyl)-3-((4-chlorophenyl)(2-(trifluoromethyl)phenyl)methoxy)azetidine-1-carboxamide is between about 20 mg to about 30 mg. In another embodiment, the amount of (R)—N-(tert-butyl)-3-((4-chlorophenyl)(2-(trifluoromethyl)phenyl)methoxy)azetidine-1-carboxamide is between about 0.5 mg to about 200 mg. In another embodiment, the amount of (R)—N-(tert-butyl)-3-((4-chlorophenyl)(2-(trifluoromethyl)phenyl)methoxy)azetidine-1-carboxamide is between about 2 mg to about 200 mg. In another embodiment, the amount of (R)—N-(tert-butyl)-3-((4-chlorophenyl)(2-(trifluoromethyl)phenyl)methoxy)azetidine-1-carboxamide is between about 5 mg to about 200 mg. In another embodiment, the amount of (R)—N-(tert-butyl)-3-((4-chlorophenyl)(2-(trifluoromethyl)phenyl)methoxy)azetidine-1-carboxamide is between about 10 mg to about 200 mg. In another embodiment, the amount of (R)—N-(tert-butyl)-3-((4-chlorophenyl)(2-(trifluoromethyl)phenyl)methoxy)azetidine-1-carboxamide is between about 15 mg to about 200 mg. In another embodiment, the amount of (R)—N-(tert-butyl)-3-((4-chlorophenyl)(2-(trifluoromethyl)phenyl)methoxy)azetidine-1-carboxamide is between about 20 mg to about 200 mg. In another embodiment, the amount of (R)—N-(tert-butyl)-3-((4-chlorophenyl)(2-(trifluoromethyl)phenyl)methoxy)azetidine-1-carboxamide is between about 25 mg to about 200 mg. In another embodiment, the amount of (R)—N-(tert-butyl)-3-((4-chlorophenyl)(2-(trifluoromethyl)phenyl)methoxy)azetidine-1-carboxamide is between about 30 mg to about 200 mg. In another embodiment, the amount of (R)—N-(tert-butyl)-3-((4-chlorophenyl)(2-(trifluoromethyl)phenyl)methoxy)azetidine-1-carboxamide is between about 35 mg to about 200 mg. In another embodiment, the amount of (R)—N-(tert-butyl)-3-((4-chlorophenyl)(2-(trifluoromethyl)phenyl)methoxy)azetidine-1-carboxamide is between about 40 mg to about 200 mg. In another embodiment, the amount of (R)—N-(tert-butyl)-3-((4-chlorophenyl)(2-(trifluoromethyl)phenyl)methoxy)azetidine-1-carboxamide is between about 45 mg to about 200 mg. In another embodiment, the amount of (R)—N-(tert-butyl)-3-((4-chlorophenyl)(2-(trifluoromethyl)phenyl)methoxy)azetidine-1-carboxamide is between about 50 mg to about 200 mg. In another embodiment, the amount of (R)—N-(tert-butyl)-3-((4-chlorophenyl)(2-(trifluoromethyl)phenyl)methoxy)azetidine-1-carboxamide. In various embodiments of the methods as described here, the amount of (R)—N-(tert-butyl)-3-((4-chlorophenyl)(2-(trifluoromethyl)phenyl)methoxy)azetidine-1-carboxamide is between about 0.1 mg to about 200 mg. In another embodiment, the amount of (R)—N-(tert-butyl)-3-((4-chlorophenyl)(2-(trifluoromethyl)phenyl)methoxy)azetidine-1-carboxamide is between about 1 mg to about 200 mg. In another embodiment, the amount of (R)—N-(tert-butyl)-3-((4-chlorophenyl)(2-(trifluoromethyl)phenyl)methoxy)azetidine-1-carboxamide is between about 10 mg to about 50 mg. In another embodiment, the amount of (R)—N-(tert-butyl)-3-((4-chlorophenyl)(2-(trifluoromethyl)phenyl)methoxy)azetidine-1-carboxamide is between about 20 mg to about 30 mg. In another embodiment, the amount of (R)—N-(tert-butyl)-3-((4-chlorophenyl)(2-(trifluoromethyl)phenyl)methoxy)azetidine-1-carboxamide is between about 0.5 mg to about 200 mg. In another embodiment, the amount of (R)—N-(tert-butyl)-3-((4-chlorophenyl)(2-(trifluoromethyl)phenyl)methoxy)azetidine-1-carboxamide is between about 2 mg to about 200 mg. In another embodiment, the amount of (R)—N-(tert-butyl)-3-((4-chlorophenyl)(2-(trifluoromethyl)phenyl)methoxy)azetidine-1-carboxamide is between about 5 mg to about 200 mg. In another embodiment, the amount of (R)—N-(tert-butyl)-3-((4-chlorophenyl)(2-(trifluoromethyl)phenyl)methoxy)azetidine-1-carboxamide is between about 10 mg to about 200 mg. In another embodiment, the amount of (R)—N-(tert-butyl)-3-((4-chlorophenyl)(2-(trifluoromethyl)phenyl)methoxy)azetidine-1-carboxamide is between about 15 mg to about 200 mg. In another embodiment, the amount of (R)—N-(tert-butyl)-3-((4-chlorophenyl)(2-(trifluoromethyl)phenyl)methoxy)azetidine-1-carboxamide is between about 20 mg to about 200 mg. In another embodiment, the amount of (R)—N-(tert-butyl)-3-((4-chlorophenyl)(2-(trifluoromethyl)phenyl)methoxy)azetidine-1-carboxamide is between about 25 mg to about 200 mg. In another embodiment, the amount of (R)—N-(tert-butyl)-3-((4-chlorophenyl)(2-(trifluoromethyl)phenyl)methoxy)azetidine-1-carboxamide is between about 30 mg to about 200 mg. In another embodiment, the amount of (R)—N-(tert-butyl)-3-((4-chlorophenyl)(2-(trifluoromethyl)phenyl)methoxy)azetidine-1-carboxamide is between about 35 mg to about 200 mg. In another embodiment, the amount of (R)—N-(tert-butyl)-3-((4-chlorophenyl)(2-(trifluoromethyl)phenyl)methoxy)azetidine-1-carboxamide is between about 40 mg to about 200 mg. In another embodiment, the amount of (R)—N-(tert-butyl)-3-((4-chlorophenyl)(2-(trifluoromethyl)phenyl)methoxy)azetidine-1-carboxamide is between about 45 mg to about 200 mg. In another embodiment, the amount of (R)—N-(tert-butyl)-3-((4-chlorophenyl)(2-(trifluoromethyl)phenyl)methoxy)azetidine-1-carboxamide is between about 50 mg to about 200 mg. In another embodiment, the amount of (R)—N-(tert-butyl)-3-((4-chlorophenyl)(2-(trifluoromethyl)phenyl)methoxy)azetidine-1-carboxamide is between about 75 mg to about 200 mg. In another embodiment, the amount of (R)—N-(tert-butyl)-3-((4-chlorophenyl)(2-(trifluoromethyl)phenyl)methoxy)azetidine-1-carboxamide is between about 100 mg to about 200 mg. In another embodiment, the amount of (R)—N-(tert-butyl)-3-((4-chlorophenyl)(2-(trifluoromethyl)phenyl)methoxy)azetidine-1-carboxamide is between about 125 mg to about 200 mg. In another embodiment, the amount of (R)—N-(tert-butyl)-3-((4-chlorophenyl)(2-(trifluoromethyl)phenyl)methoxy)azetidine-1-carboxamide is between about 150 mg to about 200 mg. In another embodiment, the amount of (R)—N-(tert-butyl)-3-((4-chlorophenyl)(2-(trifluoromethyl)phenyl)methoxy)azetidine-1-carboxamide is between about 175 mg to about 200 mg. In another embodiment, the amount of (R)—N-(tert-butyl)-3-((4-chlorophenyl)(2-(trifluoromethyl)phenyl)methoxy)azetidine-1-carboxamide is 25-500, 25-400, 25-300, 25-250, 25-200, 25-150, 25-100, or 25-75 mg. In another embodiment, the amount of (R)—N-(tert-butyl)-3-((4-chlorophenyl)(2-(trifluoromethyl)phenyl)methoxy)azetidine-1-carboxamide is 50-500, 50-400, 50-300, 50-250, 50-200, 50-150, 50-100, or 50-75 mg. In another embodiment, the amount of (R)—N-(tert-butyl)-3-((4-chlorophenyl)(2-(trifluoromethyl)phenyl)methoxy)azetidine-1-carboxamide is 75-500, 75-400, 75-300, 75-250, 75-200, 75-150, 75-100, or 75-125 mg. In another embodiment, the amount of (R)—N-(tert-butyl)-3-((4-chlorophenyl)(2-(trifluoromethyl)phenyl)methoxy)azetidine-1-carboxamide is 100-500, 100-400, 100-300, 100-250, 100-200, 100-150, 100-125 mg. In another embodiment, the amount of (R)—N-(tert-butyl)-3-((4-chlorophenyl)(2-(trifluoromethyl)phenyl)methoxy)azetidine-1-carboxamide is 1-200 mg, 10-50 mg, 10-30 mg, or 20-30 mg. In another embodiment, the amount of (R)—N-(tert-butyl)-3-((4-chlorophenyl)(2-(trifluoromethyl)phenyl)methoxy)azetidine-1-carboxamide is 5-300 mg, 5-100 mg, or 10-30 mg. In another embodiment, the amount of (R)—N-(tert-butyl)-3-((4-chlorophenyl)(2-(trifluoromethyl)phenyl)methoxy)azetidine-1-carboxamide is 1-50, 1-30, 1-25, 1-20, 1-15, 1-20, 1-5, or 1-3 mg.

Pharmaceutical compositions comprising CB1 inhibitors (e.g., compound 2) may benefit from longer half-life. In some embodiments, the CB1 inhibitor has a half-life of at least 1, 2, 3, 4, 6, 8, 10, 12, 14, 16, 18, or at least 24 hours. In some embodiments, the CB1 inhibitor has a half-life of at least 8 hours.

In some embodiments, the CB1 inhibitor (e.g., compound 2) has a beneficial safety profile. In some instances, the CB1 inhibitor has reduced seizure liability.

### Digital Processing Device

Digital processing devices may be used herein for patient monitoring. In some embodiments, the platforms, media, methods and applications described herein include a digital processing device, a processor, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing dice is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random-access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object-oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Web Browser Plug-In

In some embodiments, the computer program includes a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PUP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Additional Definitions

As used herein, “active agent” is used to indicate a chemical entity which has biological activity. In certain embodiments, an “active agent” is a compound having pharmaceutical utility. For example an active agent may be an anti-cancer therapeutic.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “therapeutically effective amount” of a chemical entity described herein refers to an amount effective, when administered to a human or non-human subject, to provide a therapeutic benefit such as amelioration of symptoms, slowing of disease progression, or prevention of disease.

“Treating” or “treatment” encompasses administration of Compound 1, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer. In some instances, treating encompasses administration of Compound 2.

As used herein, a “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salts” include, but are not limited to salts with inorganic acids, such as hydrochlorate, carbonate, phosphate, hydrogenphosphate, diphosphate, hydrobromate, sulfate, sulfinate, nitrate, and like salts; as well as salts with an organic acid, such as malate, malonate, maleate, fumarate, tartrate, succinate, citrate, acetate, lactate, gluconate, methanesulfonate, Tris (hydroxymethyl-aminomethane), p-toluenesulfonate, propionate, 2-hydroxyethylsulfonate, benzoate, salicylate, stearate, oxalate, pamoate, and alkanoate such as acetate, HOOC—(CH₂)ₙ—COOH where n is 0-4, and like salts. Other salts include sulfate, methasulfonate, bromide, trifluoroacetate, picrate, sorbate, benzilate, salicylate, nitrate, phthalate or morpholine. Pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, “subject” refers to a mammal that has been or will be the object of treatment, observation or experiment. The methods described herein can be useful in both human therapy and veterinary applications. In some embodiments, the subject is a human.

“Prodrugs” described herein include any compound that becomes Compound 1 when administered to a subject, e.g., upon metabolic processing of the prodrug. Similarly, “pharmaceutically acceptable salts” includes “prodrugs” of pharmaceutically acceptable salts. Examples of prodrugs include derivatives of functional groups, such as a carboxylic acid group, in Compound 1. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives. Other exemplary prodrugs include amides of carboxylic acids. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates. Also included are solvates formed with the one or more crystallization solvents.

Pharmaceutically acceptable forms of the compounds recited herein include pharmaceutically acceptable salts, chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range. In some instances of numerical ranges, “about” means ±10%.

As used herein, “significant” refers to any detectable change that is statistically significant in a standard parametric test of statistical significance such as Student's T-test, where p<0.05.

As used herein, the “device identifier” refers to information allowing identification of the device or a user of the device (e.g. a phone number associated with a user of a producing device). In some embodiments, the device identifier includes a phone number, email address, physical address, coordinates, IMEI number, IP address, BSSID, SSID or MAC address.

As used herein, a “user” refers to one or more person or persons associated with a system, server, or device (e.g., electronic device, member device, second device, device of a healthcare provider, etc.). Exemplary users include patients and healthcare providers (doctors, nurses, psychiatrists, pharmacists, or other provider).

As used herein, “data” refers to a collection of information about one or more entities (e.g., user of a user communication device) and/or an environment that pertains to characteristics of the one or more entities. In some embodiments, an entity is a person such as a user. In some embodiments, an entity is a thing (e.g., a house). For example, in some embodiments, data comprises sensor data from home sensors associated with a house. In this example, the data is also associated with one or more persons (e.g., the homeowner(s) and/or inhabitant(s)). In some embodiments, data refers to meta-data. In some embodiments, data comprises health information about the user of a communication device. In some embodiments, data comprises information about other users that is pre-stored in a device or in a database (e.g., a database within a group of devices who are related to the user of the user communication device as predefined by the user). In some embodiments, data comprises current data. In further embodiments, current data comprises information that is equal to or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 days old, including increments therein. In further embodiments, current data comprises information that equal to or less than 1, 2, 3, 4, 5, 6, 7, 8, 9, 10, 11, 12, 13, 14, 15, 16, 17, 18, 19, 20, 21, 22, 23, or 24 hours old. In some embodiments, data comprises historical data. In further embodiments, historical data comprises information that is equal to or more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 days old, including increments therein. In further embodiments, historical data comprises information that equal to or more than 1, 2, 3, 4, 5, 6, 7, 8, 9, 10, 11, 12, 13, 14, 15, 16, 17, 18, 19, 20, 21, 22, 23, or 24 hours old. In some embodiments, the age of information is calculated from the date the information is first collected (e.g., when a sensor first detects a sensed parameter such as, for example, heart rate).

As used herein, “health data” refers to medical information associated with a user of a device. In some embodiments, health data comprises medical history such as, for example, past illnesses, surgery, food and/or drug allergies, diseases, disorders, medical diagnostic information (e.g., genetic profile screen), or any combination thereof. In some embodiments, health data comprises family medical history (e.g., family history of breast cancer). In some embodiments, health data comprises current health information such as, for example, current symptoms, current medications, and/or current illnesses or diseases. In some embodiments, health data comprises user age, height, weight, blood type, and/or other biometrics. In some embodiments, medical history comprises medical information that is equal to or more than 1, 2, 3, 4, 5, 6, 7, 8, 9, 10, 11, 12, 13, 14, 15, 16, 17, 18, 19, 20, 21, 22, 23, or 24 hours old. In some embodiments, medical history comprises medical information that is equal to or more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days old. In some embodiments, current health information comprises information that is equal to or less than 1, 2, 3, 4, 5, 6, 7, 8, 9, 10, 11, 12, 13, 14, 15, 16, 17, 18, 19, 20, 21, 22, 23, or 24 hours old. In some embodiments, current health information comprises medical information that is equal to or less than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days old.

As used herein, “user data” refers to general information associated with a user of a device. In some embodiments, user data comprises user identity, user name, height, weight, eye color, hair color, ethnicity, national origin, religion, language(s) spoken, vision (e.g., whether user needs corrective lenses), home address, work address, occupation, family information, user contact information, emergency contact information, social security number, or any combination thereof. In some embodiments, user data is obtained via user input.

As used herein, “sensor data” refers to information obtained or provided by one or more sensors. In some instances, a sensor is associated with a device (e.g., user has a communication device with a data link via Bluetooth with a wearable sensor, such as, for example, a heart rate monitor or a pedometer). Accordingly, in some embodiments, the device obtains sensor data from the sensor (e.g., heart rate from the heart rate monitor or distance traveled from the pedometer). In some instances, the sensor data is relevant to an emergency situation (e.g., heart rate during a cardiac emergency event). In some embodiments, a sensor and/or sensor device comprises an acoustic sensor, a breathalyzer, a carbon dioxide sensor, a carbon monoxide sensor, an infrared sensor, an oxygen sensor, a pH sensor, a shock detector (e.g., on a football helmet to measure impact), a barometer, a pressure gauge, a thermometer, a proximity sensor, an occupancy sensor, or any combination thereof, and in some embodiments, sensor data comprises information obtained from any of the preceding sensors. In some embodiments, one or more sensors are physically separate from a user device. In further embodiments, the one or more sensors authorize the user device to obtain sensor data. In further embodiments, the one or more sensors provide or send sensor data to the user device autonomously. In some embodiments, the user device and the one or more sensors belong to the same group of devices, wherein member devices are authorized to share data. In some embodiments, a user device comprises one or more sensors (e.g., user device is a wearable device having a sensor or sensing component).

Modern communication devices, for example, smart phones, tablet computers, wearable communication devices, smart sensor devices and/or systems are often equipped with a variety of features for determining location information of the communication device using, for example, GPS, or triangulation with cellular phone towers. Modern communication devices also often include functionality to store data regarding a user of the communication device, for example, health information about the user.

Reference in the present specification to an “alkyl” group means a branched or unbranched, cyclic or acyclic, saturated or unsaturated (e.g. alkenyl (including allyl) or alkynyl (including propargyl)) hydrocarbyl radical. Where cyclic or acyclic the alkyl group is preferably C₁ to C₁₂, more preferably C₁ to C₈ (such as methyl, ethyl, propyl, isopropyl, butyl, isobutyl, tert-butyl, sec-butyl, pentyl, isopentyl, hexyl, heptyl, octyl). It will be appreciated therefore that the term “alkyl” as used herein includes alkyl (branched or unbranched), alkenyl (branched or unbranched), alkynyl (branched or unbranched), cycloalkyl, cycloalkenyl and cycloalkynyl. A cyclic alkyl group may also be a mono-bridged or multiply-bridged cyclic alkyl group. In a preferred embodiment, a cyclic alkyl group is preferably C₃ to C₁₂, more preferably C₅ to C₈ and an acyclic alkyl group is preferably C₁ to C₁₀, more preferably C₁ to C₆, more preferably methyl, ethyl, propyl (n-propyl or isopropyl), butyl (n-butyl, isobutyl, tertiary butyl or sec-butyl) or pentyl (including n-pentyl and iso-pentyl), more preferably methyl.

As used herein, the term “lower alkyl” means a branched or unbranched, cyclic or acyclic, saturated or unsaturated (e.g. alkenyl or alkynyl) hydrocarbyl radical wherein said cyclic lower alkyl group is C₅, C₆ or C₇, and wherein said acyclic lower alkyl group is C₁, C₂, C₃ or C₄. It will be appreciated therefore that the term “lower alkyl” as used herein includes lower alkyl (branched or unbranched), lower alkenyl (branched or unbranched), lower alkynyl (branched or unbranched), cycloloweralkyl, cycloloweralkenyl and cycloloweralkynyl. Preferably, a lower alkyl group is preferably selected from methyl, ethyl, propyl (n-propyl or isopropyl) or butyl (n-butyl, isobutyl, tertiary-butyl or sec-butyl), more preferably methyl.

Reference in the present specification to an “aryl” group means a mono or bicyclic aromatic group, such as phenyl or naphthyl, and preferably a mono-cyclic aromatic group.

Reference in the present specification to a “heteroaryl” group means an aromatic group containing one or more heteroatoms, preferably 1, 2 or 3 heteroatoms, preferably 1 or 2 heteroatoms. Preferably the heteroatoms are selected from O, S and N, preferably from O and N. Preferably the heteroaryl group comprises 5 or 6-membered ring systems. The heteroaryl group is preferably a monocyclic or bicyclic ring system, preferably monocyclic. Examples include thienyl, furanyl, pyrrolyl, imidazolyl, pyrazolyl, thiazolyl, isothiazolyl, oxazolyl, isoxazolyl, pyridyl, pyrazinyl, pyrimidinyl, quinolinyl, isoquinolinyl, benzofuranyl and isobenzofuryl.

Reference in the present specification to a non-aromatic heterocyclic group is to a saturated or partially unsaturated 4, 5, 6 or 7-membered ring containing 1, 2 or 3 heteroatoms selected from N, O and S, preferably 1 or 2 heteroatoms, preferably selected from N and O. Examples include piperidinyl, morpholinyl, piperazinyl and pyrrolidinyl.

The alkyl, aryl and heteroaryl groups may be substituted or unsubstituted. In one embodiment, only the alkyl, aryl and heteroaryl groups defined herein as R¹ to R¹⁶ may be substituted. Where R⁷ and R⁸ together form a 5 or 6-membered ring, the ring may be substituted or unsubstituted. Where substituted, there will generally be 1 to 3 substituents present, preferably 1 or 2 substituents. Substituents may include:
carbon containing groups such as alkyl aryl, arylalkyl (e.g. substituted and unsubstituted phenyl, substituted and unsubstituted benzyl);
halogen atoms and halogen containing groups such as haloalkyl (e.g. trifluoromethyl);
oxygen containing groups such as alcohols (e.g. hydroxy, hydroxyalkyl, (aryl)(hydroxy)alkyl), ethers (e.g. alkoxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arylcarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl) and amides (e.g. aminocarbonyl, mono- or dialkylaminocarbonyl, aminocarbonylalkyl, mono- or dialkylaminocarbonylalkyl, arylaminocarbonyl);
nitrogen containing groups such as amines (e.g. amino, mono- or dialkylamino, aminoalkyl, mono- or dialkylaminoalkyl), azides, nitriles (e.g. cyano, cyanoalkyl), nitro; sulphur containing groups such as thiols, thioethers, sulphoxides and sulphones (e.g. alkylthio, alkylsulfinyl, alkylsufonyl, alkylthioalkyl, alkylsulfinylalkyl, alkylsulfonylalkyl, arylthio, arylsulfinyl, arylsulfonyl, arylthioalkyl, arylsulfinylalkyl, arylsulfonylalkyl); and
heterocyclic groups containing one or more, preferably one, heteroatom, (e.g. thienyl, furanyl, pyrrolyl, imidazolyl, pyrazolyl, thiazolyl, isothiazolyl, oxazolyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, piperazinyl, morpholinyl, thionaphthyl, benzofuranyl, isobenzofuryl, indolyl, oxyindolyl, isoindolyl, indazolyl, indolinyl, 7-azaindolyl, isoindazolyl, benzopyranyl, coumarinyl, isocoumarinyl, quinolyl, isoquinolyl, naphthridinyl, cinnolinyl, quinazolinyl, pyridopyridyl, benzoxazinyl, quinoxadinyl, chromenyl, chromanyl, isochromanyl and carbolinyl).

Where an aryl group is phenyl, the phenyl may be substituted by adjacent substituents forming a 5 or 6 membered saturated ring optionally containing 1 or 2 heteroatoms, preferably selected from N, O and S, preferably from N and O. Where the saturated ring contains 2 nitrogen atoms, the ring is preferably a 6-membered ring. Where the saturated ring contains 2 oxygen atoms, the ring may be a 5- or 6-membered ring. Examples include 2,3-dihydrobenzo[b]furan-7-yl, 2,3-dihydrobenzo[b]thiophen-6-yl, 1,2,3,4-tetrahydronaphthalen-5-yl, 2,3-dihydrobenzo[1,4]dioxin-6-yl and 1,2,3,4-tetrahydroisoquinolin-8-yl.

Preferred substituents include alkyl (including haloalkyl), alkoxy (including haloalkoxy), aryl, nitrile or halo. Preferred halogen-containing groups include trifluoromethyl.

As used herein, the term “alkoxy” means alkyl-O— and “alkoyl” means alkyl-CO—.

As used herein, the term “halogen” means a fluorine, chlorine, bromine or iodine radical, preferably a fluorine or chlorine radical.

### EXAMPLES

The following examples serve to further describe the manner of using the present disclosure. These examples are presented for illustrative purpose and should not serve to limit the true scope of the present disclosure.

In carrying out the procedures of the methods described herein, it is of course to be understood that references to particular buffers, media, reagents, cells, culture conditions, and the like are not intended to be limiting, but are to be read so as to include all related materials that one of ordinary skill in the art would recognize as being of interest or value in the particular context in which that discussion is presented. For example, it is often possible to substitute on buffer system or culture medium for another and still achieve similar, if not identical, results. Those of skill in the art will have sufficient knowledge of such systems and methodologies so as to able, without undue experimentation, to make such substitutions as will optimally serve their purposes in using the methods and procedures disclosed herein.

### Example 1—Use of Compound 2 for Treating CUD

Compound 2 is used in a combination treatment for cannabis use disorder (CUD). Combinations include use of antidepressants (specifically sedating antidepressants, such as mirtazapine or trazodone), antipsychotics, CB1 agonists such as THC and allosteric CB1 modulators such as Cannabidiol. These agents improve the safety profile of a CB1 inhibitor as described herein, such as compound 2, and lessen the burden of the withdrawal symptoms.

Compound 2 has several unique properties, such as:
Long half-life, where accumulation enables slow onset, even absent a titration schedule. In steady state, the patient has 24 h coverage despite convenient once daily dosing. Additionally, due to the long half-life, occasional skipped doses are less of a problem which should improve efficacy/reduce the risk of relapse.
Lack of drug-drug interactions, particularly CYP3A4 inhibition. Both Trazodone and Mirtazapine are metabolized by CYP3A4 and it has been shown that CYP3A4 inhibitors increase the concentrations of both drugs.
Lack of seizure liability. Both antidepressants and drug withdrawal can decrease the seizure threshold.

The benefits of a combination therapy comprising compound 2 and a sedating antidepressant, such as Mirtazapine or Trazodone, are outline in Table 1 below.

TABLE 1
Benefits of Combination Therapy Comprising
Compound 2 and a Sedating Antidepressant

| | | Effect of sedating | Total Effect and |
|---|---|---|---|
| Cannabis Withdrawal | Effect of Compound 2 | antidepressant | Benefits |
| High probability of | Blocks getting high + | | Reduced |
| THC use/relapse | has anti-addictive | | probability of |
| | properties | | relapse, improved |
| | | | cognition and |
| | | | better social and |
| | | | professional |
| | | | outcomes |
| Increased anxiety | Increases anxiety | Decreases anxiety | Reduced anxiety |
| (Withdrawal | | | |
| symptoms) | | | |
| Sleep problems | Increases sleep | Induces Sleep | Improved sleep |
| (Withdrawal | problems | | |
| symptoms) | | | |
| | Depressed mood | Improves mood | Improved mood |
| | Weight loss | Weight gain | Neutral effect on |
| | | | weight |

Daily doses such as between about 5 mg and about 100 mg of Compound 2 are efficacious in CUD. Doses of 1-50 mg or 20 to 30 mg may also be used. Compound 2 is also evaluated in combination with daily doses of Mirtazapine at about 15 mg to about 45 mg, or daily doses of Trazodone at about 50 mg to about 400 mg. Mirtazapine is given at bedtime. In another embodiment, Compound 2 and Mirtazapine are formulated and administered as a single oral dosage form, such as a tablet or a capsule.

In order to improve tolerability and adherence in some circumstances, a titration scheme is used, as outlined in Table 2 below.

TABLE 2
Titration Scheme of Compound 2

| Week 1 | Week 2 | Week 3 and beyond |
|---|---|---|
| 5 mg | 10 mg | 20 mg |
| 10 mg | 20 mg | 50 mg |
| 20 mg | 50 mg | 100 mg |

Chronic dosing after several weeks or months of abstinence with low dose, such as about 5 mg to about 50 mg, is used to prevent relapses.

The doses of compound 2 described are combined with about 15 to about 45 mg of Mirtazapine, or another sedating antidepressant such as Trazodone, or a CB1 agonist such as THC, or an allosteric CB1 modulator such as Cannabidiol.

In another embodiment, to prevent mix ups and improve compliance, the pills or capsules are packaged in blisters representing one week each. To prevent potential accidental or suicidal overdoses, weekly blisters are handed out by the treating physician or another healthcare professional/pharmacist.

### Example 2—Technology Based Monitoring of Patients

A patient is diagnosed with an addictive disorder and treated with a CB1 inhibitor described herein (e.g., Compound 2). Additionally, the patient is enrolled in a mobile application which provides monitoring during the course of treatment. The mobile application is accessed via phone, computer, smart watch, or other device and provides a user interface for the client to login with unique credentials such as a username and password, or biometric authorization (e.g., retinal scan, facial identification, or fingerprint). Once logged in, the patient is presented with an interface describing one or more of previously submitted health reports, reminders to take medication, current health/vital statistics, contact information of healthcare provides, and reminders to submit health questionnaires. In some instances, the mobile application is populated with patient information from an EMR (electronic medical record). In some instances, a questionnaires is completed prior to the start of treatment to establish a baseline. Additional health statistics may then be acquired during treatment, such as heart rate, skin conductance, temperature, sleep quality, sleep duration, blood pressure, or other biological metric obtained from a sensor either integrated or external to the device comprising the mobile application.

As the treatment progresses, the patient is reminded to submit health reports to his/her provider (primary care physician, physiatrist, or other healthcare professional) for evaluation. The information may be processed and evaluated by software for changes which are associated with negative health outcomes, such as depression or self-harm. The information may be evaluated by the healthcare provider to determine if treatment should be continued, suspended, or discontinued. The healthcare provider may additionally recommend additional changes to the treatment such as changes in dosage, frequency, of the CB1 inhibitor, and/or changes in dosage, frequency of an antidepressant drug if applicable. In an example, a patient's amount of sleep increases by an amount (e.g., 30%) from baseline levels after treatment, and the healthcare provider is notified. In some instances, the notification recommends a follow-up appointment with the patient for further evaluation. In some instances, the mobile application is configured to arrange a telehealth visit directly with the provider.

In another embodiment, to prevent mix ups and improve compliance, the pills or capsules are packaged in blisters representing one week each. To prevent potential accidental or suicidal overdoses, weekly blisters are handed out by the treating physician or another healthcare professional/pharmacist. Mood is monitored remotely, for instance through the mobile application and the blister pack comprising the treatment for the next week is only delivered to the patient if the patient has filled out the questionnaire but does not show any signs of an emerging mood disorder or other psychiatric/behavioral symptoms that could hint at an increased risk of suicidality or self-harm.

While some embodiments have been shown and described, various modifications and substitutions may be made thereto without departing from the spirit and scope of the present disclosure. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present disclosure has been described by way of illustration and not limitations on the scope of the claims.

What is claimed is:

1. A method of treating an addictive disorder comprising administering to a patient:
a. a first treatment comprising a pharmaceutical composition, wherein the pharmaceutical composition comprises an effective amount of a CB1 inhibitor; and
b. a second treatment.

2. The method of claim 1, wherein the addictive disorder comprises a substance addiction.

3. The method of claim 1, wherein the addictive disorder comprises a behavioral addiction.

4. The method of claim 1, wherein the addictive disorder comprises addiction to one or more of cannabis, alcohol, nicotine, opioids, amphetamines, cocaine, and gambling.

5. The method of claim 1 or 2, wherein the addictive disorder comprises cannabis use disorder (CUD).

6. The method of claim 1 or 2, wherein the addictive disorder comprises cannabis hyperemesis syndrome (HES).

7. The method of any one of claims 1-6, wherein the second treatment comprises one or more of an antidepressant, CB1 agonist, CB1 modulator, and patient monitoring.

8. The method of claim 7, wherein the patient monitoring comprises use of a software application.

9. The method of claim 7, wherein the patient monitoring comprises evaluation by a healthcare professional.

10. The method of claim 8, wherein the software application is configured for use on mobile phones, tablets, watches, or wristbands.

11. The method of any one of claims 8-10, wherein the software application is configured to evaluate the patient's mood or mental state.

12. The method of any one of claims 8-10, wherein the software application is configured to evaluate the patient's vital signs, speech, movement, or sleep patterns.

13. The method of any one of claims 8-11, wherein the software application comprises a patient questionnaire.

14. The method of any one of claims 8-13, wherein the software application comprises monitoring the patient's vital signs.

15. The method of claim 7, wherein the CB1 agonist comprises THC or other agonist derived from the cannabis plant.

16. The method of claim 7, wherein the CB1 modulator comprises cannabidiol, Org27569 PAM1 (GAT211), ZCZ011, ABD1027, PSNCBAM-1, GAT358, Lipoxin A4, Pregnenolone, or Pepcan 12.

17. The method of claim 7, wherein the antidepressant comprises a selective serotonin reuptake inhibitor (SSRI), a serotonin and norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine-dopamine reuptake inhibitor (NDRI), a monoamine oxidase inhibitor (MAOI), a tetracyclic antidepressant (TeCA), an atypical antipsychotic, a tricyclic antidepressant (TCA), an alternative antidepressant, or an over-the-counter antidepressant.

18. The method of claim 17, wherein the SSRI comprises citalopram, escitalopram, fluoxetine, fluvoxamine, paroxetine, or sertraline.

19. The method of claim 17, wherein the SNRI comprises desvenlafaxine, duloxetine, levomilnacipran, milnacipran, or venlafaxine.

20. The method of claim 17, wherein the SMS comprises vilazodone or vortioxetine.

21. The method of claim 17, wherein the SARI comprises nefazodone or trazodone.

22. The method of claim 17, wherein the NRI comprises reboxetine, teniloxazine, viloxazine, or atomoxetine.

23. The method of claim 17, wherein the MAOI comprises isocarboxazid, nialamide, phenelzine, hydracarbazine, tranylcypromirie, bifemelane, moclobemide, pirlindole, toloxatone, rasagiline, selegilin, caroxazone, or safinamide.

24. The method of claim 17, wherein the TeCA comprises amoxapine, maprotiline, mianserin, mirtazapine, or setiptiline.

25. The method of claim 17, wherein the TCA comprises amitriptyline, amitriptylinoxide, clomipramine, desipramine, dibenzepin, dimetacrine, dosulepin, doxepin, imipramine, lofepramine, melitracen, nitroxazepine, nortriptyline, noxiptiline, opipramol, pipofezine, protriptyline, or trimipramine.

26. The method of claim 17, wherein the atypical antipsychotic comprises amisulpride, lurasidone, or quetiapine.

27. The method of claim 17, wherein the alternative antidepressant comprises agomelatine, ketamine, tandospirone, tianeptine, and minocycline.

28. The method of claim 17, wherein the over-the-counter antidepressant comprises ademetionine, Hypericum perforatum, oxitriptan, rubidium chloride, or tryptophan.

29. The method of any one of claims 7-28, wherein the antidepressant and the CB1 inhibitor are metabolized by different enzymes in-vivo.

30. The method of claim 29, wherein the CB1 inhibitor is not an inhibitor of CYP3A4.

31. The method of claim 29, wherein the CB1 inhibitor is metabolized by one or more of CYP3A4, CYP2C9, CYP2C19, CYP1A2, CYP2E1, CYP2D6, and CYP2A6.

32. The method of claim 29, wherein the CB1 inhibitor comprises an IC₅₀ of individual CYP450s of less than 40 micromolar in human liver microsomes.

33. The method of claim 29, wherein the antidepressant is metabolized primarily by CYP3A4.

34. The method of any one of claims 7-33, wherein the dose of the antidepressant is 0.1 mg to 1000 mg.

35. The method of claim 24, wherein the dose of the antidepressant is 15 mg to 45 mg.

36. The method of any one of claims 1-34, wherein the CB1 inhibitor comprises an antagonist, inverse agonist, or reverse agonist.

37. The method of claim 36, wherein the CB1 inhibitor has a half-life of at least 2 hours.

38. The method of claim 36, wherein the CB1 inhibitor has a half-life of at least 8 hours.

39. The method of claim 36 or 37, wherein the CB1 inhibitor has a reduced seizure liability.

40. The method of claim 36, wherein the CB1 inhibitor comprises rimonabant, taranabant, MK-0364, AM251, AM1387, AM4113, cannabigerol, ibipinabant, otenabant, surinabant, tetrahydrocannabivarin and virodamine, TM-38837, AM6545, or a CB1 targeting-antibody.

41. The method of any one of claims 1-39, wherein the CB1 inhibitor has the structure of formula (I): 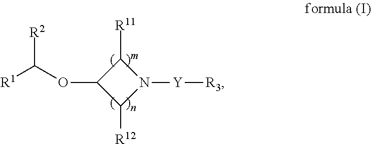
or a pharmaceutically acceptable salt or prodrug thereof,
wherein
R¹ is aryl or heteroaryl;
R² is alkyl, aryl or heteroaryl;
R³ is alkyl, aryl, heteroaryl, NR⁹R¹⁰, OR¹⁵, or NR¹⁶C(O)R¹⁷;
Y is C═O, C═S, SO₂, or (CR⁷R⁸)ₚ;
R⁷ and R⁸ are independently selected from H and lower alkyl;
R⁹ is selected from H, alkyl, aryl, heteroaryl, and non-aromatic heterocyclic groups, or together with R¹⁰ forms a saturated 4, 5, 6, or 7 membered ring optionally containing an additional heteroatom selected from N and O;
R¹⁰ is selected from H and lower alkyl, or together with R⁹ forms a saturated 4, 5, 6, or 7 membered ring optionally containing an additional heteroatom selected from N and O;
R¹¹ and R¹² are independently selected from H and lower alkyl;
R¹⁵ is selected from alkyl and aryl;
R¹⁶ is selected from H and lower alkyl;
R¹⁷ is selected from alkyl, aryl and heteroaryl;
m is 1 or 2;
n is 1 or 2; and
p is 1, 2, 3 or 4.

42. The method of claim 41, wherein m is 1 and n is 1.

43. The method of claim 41 or 42, wherein R¹ and R² are independently aryl.

44. The method of any one of claims 41-43, wherein at least one of R¹ and R² has a non-hydrogen substituent in the ortho-position(s) thereof relative to the point of attachment to the [—CH—O—] group.

45. The method of any one of claims 41-44, wherein R¹¹ and R¹² are hydrogen.

46. The method of any one of claims 41-45, wherein R³ is NR⁹R¹⁰, and R⁹ and R¹⁰ are independently lower alkyl or hydrogen.

47. The method of claim 41, wherein the CB1 inhibitor comprises the structure of formula (Ia): 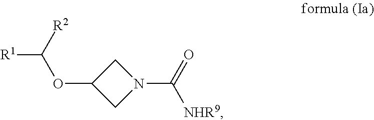
or a pharmaceutically acceptable salt or prodrug thereof.

48. The method of claim 47, wherein R¹ and R² are independently selected from a group of formula (II): 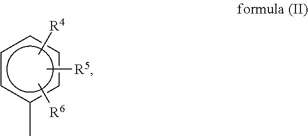
wherein
R⁴, R⁵, and R⁶ are independently selected from hydrogen, halo, alkyl (including haloalkyl), thioalkyl, alkoxy (including haloalkoxy), alkylsulfonyl, amino, mono- and di-alkyl amino, mono- and di-aryl amino, alkylarylamino, aminoalkyl, alkylaminoalkyl, dialkylaminoalkyl, NR¹⁴C(O)R¹⁹, NR¹⁴SO₂R²⁰, COOR¹⁹, OC(O)R²⁰, CONR¹³R¹⁴ and SO₂NR¹³R¹⁴,
R¹³ and R¹⁴ are independently selected from hydrogen and alkyl or form a 5 or 6 membered ring optionally containing 1 or 2 additional heteroatoms selected from N, O and S;
R¹⁹ is selected from H, alkyl, aryl and heteroaryl, and
R²⁰ is selected from alkyl, aryl and heteroaryl.

49. The method of claim 48, wherein at least one of R⁴, R⁵, and R⁶ are chloro or trifluoromethyl.

50. The method of claim 41, wherein the CB1 inhibitor has the structure:

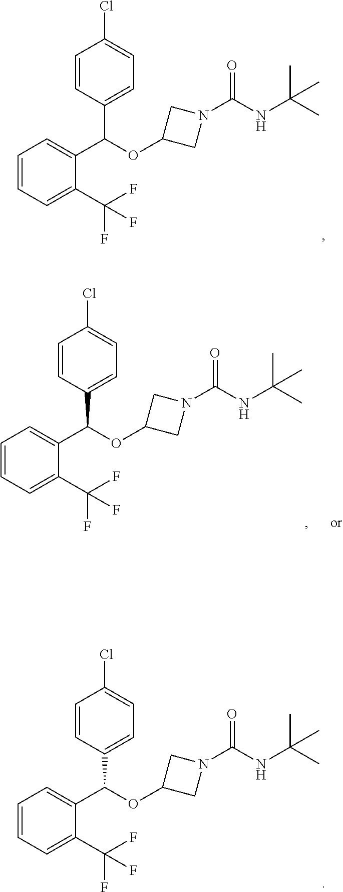 [SMILES: CC(C)(C)NC(=O)N1CC(OC(c2ccc(Cl)cc2)c2ccccc2C(F)(F)F)C1 ; CC(C)(C)NC(=O)N1CC(O[C@H](c2ccc(Cl)cc2)c2ccccc2C(F)(F)F)C1 ; CC(C)(C)NC(=O)N1CC(O[C@@H](c2ccc(Cl)cc2)c2ccccc2C(F)(F)F)C1]

51. The method of any one of claims 1-50, wherein the pharmaceutical composition is prepared as an oral, sublingual, buccal, rectal, nasal, or parenteral dose.

52. The method of any one of claims 1-50, wherein the dose of the CB1 inhibitor is 1 mg to 200 mg.

53. The method of claim 52, wherein the dose of the CB1 inhibitor is 1-50 mg, 5 mg to 100 mg, or 20-30 mg.

54. The method of any one of claims 1-53, wherein the CB1 inhibitor is formulated as an oral, parenteral, intravenous (IV), intramuscular (IM), subcutaneous (SC), endotracheal, sublingual, buccal, intralingual, submental, transdermal, suppository, or intranasal administration.

55. The method of any one of claims 1-54, further comprising administering a pharmaceutically acceptable alkaline agent.

56. The method of any one of claims 1-55, wherein the pharmaceutical composition is formulated to deliver an effective dose of the CB1 inhibitor in no more than 10 min.

57. The method of any one of claims 1-56, wherein the CB1 inhibitor is dosed daily.

58. The method of any one of claims 17-57, wherein the antidepressant is dosed daily.

59. A pharmaceutical composition comprising the CB1 inhibitor of any one of claims 1-56 and an antidepressant of any one of claims 17-56.

60. The pharmaceutical composition of claim 59, wherein the pharmaceutical composition is formulated as a capsule or a tablet.
